(19) United States
(12) Patent Application Publication
(10) Pub. No.: US 2014/0172314 A1
(43) Pub. Date: Jun. 19, 2014

(54) SYSTEMS AND METHODS FOR DETERMINING CALORIC INTAKE USING A PERSONAL CORRELATION FACTOR

(71) Applicant: Access Business Group International LLC, Ada, MI (US)

(72) Inventors: David W. Baarman, Fennville, MI (US); Matthew K. Runyon, East Grand Rapids, MI (US); Cody D. Dean, Grand Rapids, MI (US); Neil W. Kuyvenhoven, Ada, MI (US); Sheri A. Hunt, Manhattan Beach, CA (US); Rodney A. Velliquette, Ada, MI (US)

(73) Assignee: Access Business Group International LLC, Ada, MI (US)

(21) Appl. No.: 14/087,025

(22) Filed: Nov. 22, 2013

Related U.S. Application Data
(60) Provisional application No. 61/739,384, filed on Dec. 19, 2012.

(51) Int. Cl. A61B 5/00

(52) U.S. Cl. CPC A61B 5/4866; A61B 5/4872
USPC 702/19

(57) ABSTRACT

Systems and methods are provided for determining an individual's personal correlation factor and, using the personal correlation factor, determining the individual's caloric intake. A method for determining a personal correlation factor includes determining a body composition change over a calibration period, converting the body composition change to an equivalent energy value, and dividing the equivalent energy value by a net caloric value for the same calibration period, wherein the net caloric value includes a caloric expenditure less a caloric intake. A method for determining a subsequent caloric intake includes converting a body composition change to an equivalent energy value, dividing the equivalent energy value by the personal correlation value, and adding to this quotient the individual's caloric expenditure, wherein each step is performed using a processor.

### BACKGROUND OF THE INVENTION

The present invention relates to systems and methods for determining an individual's caloric intake using a personal correlation factor.

An increased public interest in healthy living has resulted in a growing market for personal fitness aids. For example, so-called pocket pedometers are increasingly popular among fitness enthusiasts, and approximate calories and fat grams burned over the course of a workout. Pedometers are also available as a download to a smart phone or tablet computer. These fitness aids can additionally provide workout logs and can suggest aerobic exercises and varied workout routines.

Current fitness aids also provide an approximation of an individual's caloric intake based on a manual food log. However, non-entries or incorrect entries to the food log can often lead to an under-approximated caloric intake. As a result, an individual can experience a weight gain when a weight loss is expected, despite logging a caloric intake less than the total amount of calories burned.

Accordingly, there remains a continued need for an improved determination of an individual's caloric intake. In particular, there remains a continued need for an improved determination of an individual's caloric intake that can be used in conjunction with a measured energy expenditure for weight loss programs, weight management programs, and general health and fitness programs.

### SUMMARY OF THE INVENTION

Systems and methods for determining caloric intake are provided. The systems and methods include determining an individual's personal correlation factor and, using the personal correlation factor, determining the individual's caloric intake. The caloric intake can be used in conjunction with a weight loss or weight management program and for other purposes.

In one embodiment, a method for determining a personal correlation factor for an individual is provided. The method includes determining a body composition change over a calibration period, converting the body composition change to an equivalent energy value, and dividing the equivalent energy value by a net caloric value for the same calibration period, wherein the net caloric value includes the individual's caloric expenditure less the individual's caloric intake.

In another embodiment, the body composition change is determined using a bio-impedance sensor, and the caloric expenditure is determined using a pedometer. The caloric intake can be measured based on a food log for only the calibration period. Thereafter, the personal correlation factor can be used to indirectly measure caloric intake, without requiring use of the food log.

In still another embodiment, a wearable device is provided. The wearable device uses the individual's personal correlation factor to determine caloric intake, and to suggest an activity adjustment and/or a dietary adjustment. The wearable device includes a first sensor configured to measure the wearer's caloric expenditure, a second sensor configured to measure the wearer's body composition, a memory adapted to store the wearer's personal correlation factor, and a processor electrically coupled to the first and second sensors and adapted to perform a computer operation to determine the individual's caloric intake. The first sensor includes a pedometer or an accelerometer, and the second sensor includes a bio-impedance sensor. The wearable device is self-contained within a housing and worn on the wearer's wrist, ankles, or hips, for example.

In even another embodiment, a method for determining an individual's caloric intake using that individual's personal correlation factor is provided. The method includes converting a body composition change to an equivalent energy value, dividing the equivalent energy value by the personal correlation value, and adding to this quotient the individual's caloric expenditure, wherein each step is performed using a processor. The method can additionally include reporting the caloric intake, optionally with reference to a target value. Still further optionally, the method can include recommending a dietary modification and/or recommending an exercise regimen in response to the determined caloric intake.

These and other features and advantages of the present invention will become apparent from the following description of the invention in accordance with the accompanying drawings and appended claims.

### BRIEF DESCRIPTION OF THE DRAWINGS

FIG. 1 shows the flow of current through the body during a bio-impedance spectroscopy measurement.

FIG. 2 includes expressions used in the derivation of a personal correlation factor.

FIG. 3 is a flow chart illustrating a method for determining a personal correlation factor in accordance with an embodiment of the invention.

FIG. 4 is a four week calibration log for determining a personal correlation factor.

FIG. 5 is an exemplary look-up table for personal correlation values by age, gender, and workout frequency.

FIG. 6 is a flow chart illustrating a method for determining caloric intake using a personal correlation factor in accordance with an embodiment of the invention.

FIG. 7 is schematic diagram of a wearable device for performing the method of FIG. 6.

FIG. 8 illustrates the transfer of information from a wearable body composition and activity measurement device to a computer.

### DETAILED DESCRIPTION OF THE CURRENT EMBODIMENTS

The invention as contemplated and disclosed herein includes systems and methods for determining an individual's personal correlation factor and, using the personal correlation factor, determining the individual's caloric intake. Part I includes an overview of the relationship between caloric intake, caloric expenditure, stored body mass, and a personal correlation factor. Part II includes systems and methods for determining an individual's personal correlation factor. Part III includes systems and methods for determining the individual's caloric intake using the personal correlation factor to assist the individual in meeting his or her weight management goals.

### I. Overview

The management of energy in the human body can be modeled by equation (1) below, where I(t) is the total caloric intake, E(t) is the total caloric expenditure, and U(t) is the stored caloric value:

I(t)−E(t)=U(t) (1)

According to the above equation, the caloric intake less the caloric expenditure is equal to the stored caloric value. Where the caloric intake is greater than the caloric expenditure, the stored caloric value is positive. Where the caloric intake is less than the caloric expenditure, the stored caloric value is negative.

The caloric expenditure E(t) in equation (1) can be further defined by equation (2) below, where BMR is the basal metabolic rate, AIE is the activity induced expenditure, TEF is the thermal effect of food, and NEAT is the non-exercise activity thermogenesis:

E(t)=BMR+AIE+TEF+NEAT (2)

BMR is a clinical measurement that can be measured while the individual is completely stationary, and is typically performed in a clinical setting. The individual's resting metabolic rate RMR is an approximation for BMR, and gives more leeway to small movements while measuring. Equations (3) and (4) below provide a predictive value for an individual's RMR, which again is used in place of BMR in equation (2) above:

Men: RMR=9.99·weight+6.25·height·4.92−age+5 (3)

Women: RMR=9.99·weight+6.25·height·4.92·age−161 (4)

Referring again to equation (2) for the energy expenditure E(t), the individual's activity induced expenditure AIE is determined based on certain physical characteristics and data collected by a 3-axis accelerometer. For example, speed may be calculated using equation (5):

Speed(m/min)=26.82×(SMA−1.34)×(249−7.86×H+0.0614×(H)²+3.05×H×A−0.00907×W×NC−0.0671×A×NC)+2.81 (5)

From equation (5) above, H is height, NC is the number of times the individual performs cardio, A is age, and W is the individual's weight in pounds.

Another component of AIE is VO₂, which is a measure of the rate at which a person's body uses or transports oxygen. Equation (6) below from the American College of Sports Medicine (ACSM) can be used to estimate VO₂:

VO₂=α₁·S+β₁·S·G

VO₂ can be expressed in liters per minute, or as a rate per unit mass of the person such as milliliters per kilogram per minute. In equation (6) there are three parts, horizontal, vertical, and resting. Resting is left out because it is addressed above. The horizontal portion is the first part of equation (6). The α₁ term is constant, and S is the speed the person is moving in meters per minute from equation (5) above. The second portion is the vertical piece where β₁ is a constant S is speed, and G is the gradient of the hill.

Another way to estimate VO₂ is identified below in Equation (7) below:

VO₂=αₙS+βₙ·S·G+F(GP,A,S) (7)

The first part of equation (7) is similar to equation (6), however, the coefficients change depending on what segment of speed the individual is moving at. If the individual is walking, these coefficients are different from when the individual is running. These coefficients can be expressed as a function of speed as shown in equation (8) and (9),

αₙ=a·S+b, (8)

βₙ=c·S+d, (9)

where a, b, c, and d are constants. Substituting these equations into the first portion of equation (7) results in a multivariable polynomial equation (10):

VO₂=a·S²+b·S+c·S²·G+d·S·G+F(GP,A,S)+ε (10)

From equation (10) above, ε is an error term, and F(GP, A,S) is a function of genetic profile, age, and sex. This function can make the calculations specific to the individual. Each individual takes in a different amount of oxygen when working out, and according to the ACSM equation's two people weighing the same will have the same VO₂ levels. However, this is typically not the case. For example, an out of shape 130 lb. male child will burn energy at a different rate than a 130 lb. female marathon runner.

Equation (10) uses the following conversion equation (11) to calculate AIE. It is based on the premise that the average person burns 5 kcal per liter of O₂.

$$AIE = {VO}_{2} \cdot Weighted ( lbs ) \cdot \frac{2.2}{1000} \cdot \frac{5 kcals}{{LiterO}_{2}} \qquad ( 11 )$$

The thermic effect of food (TEF) portion of equation (2) for calculating E(t) is based on the number of calories consumed in a day. An accepted approximation for TEF is given below in equation (12):

TEF=0.075·I(t) (12)

Referring again to equation (2) for energy expenditure, NEAT is a fixed value based on a person's lifestyle. Whatever is not quantified from the AIE in equation (11) can be rolled into NEAT using activity codes and Metabolic Equivalent Task (MET) intensities. If I(t) is unknown, NEAT may be ignored from equation (2).

Referring again to equation (1), U(t) is the change in energy stored (positive) or used (negative) by the body. This energy is stored either as fat mass or fat free mass. One method for determining the individual's body composition (i.e., the component fat mass and the component fat free mass) includes underwater weighing and water displacement tests. This measurement technique requires the individual to be fully submersed in a tank of water and measuring both the underwater weight and the change in water volume change upon submersion. These two measurements are then used to calculate body fat percentage. This method requires trained personnel and is not easily performed, however.

Other methods for determining body fat percentage include bio-impedance analysis (BIA) and bio-impedance spectroscopy (BIS). Bio-impedance analysis is performed by applying a low alternating current (˜800 μA) across two points on the body and measuring the complex impedance to the flow of current. Complex impedance is composed of a resistance, R (Ohms) and a reactance, Xc (Ohms). This type of analysis can be performed at single or multiple frequencies. Single frequency BIA is performed at 50 kHz and multi-frequency BIA is typically performed at seven discrete frequencies between a 0 kHz and 500 kHz (up to 1000 kHz).

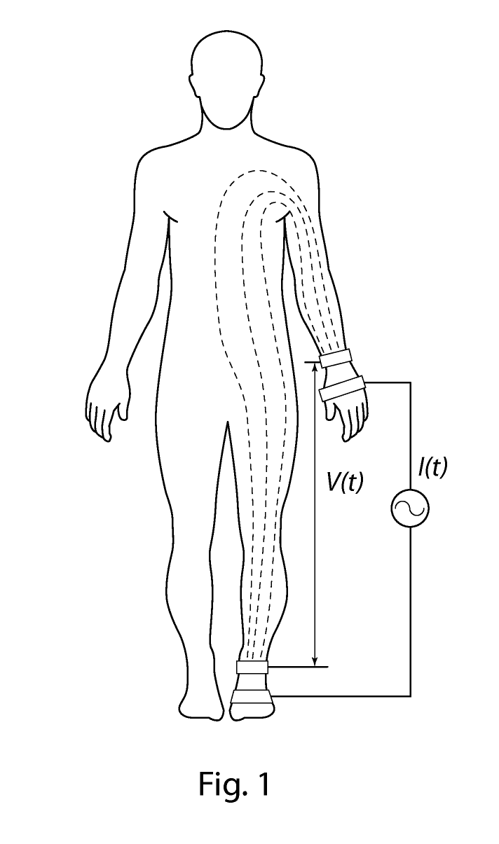

BIS is similar to multi-frequency BIA, except BIS measures up to 256 discrete frequencies between 0 kHz and 1000 kHz. For example, FIG. 1 illustrates the flow of current through the body during a BIS frequency sweep. The raw impedance data from BIS frequency sweeps is converted to Xc versus R plot to determine two characteristic resistance values. The first characteristic resistance value, R₀, is the resistance value obtained when frequency is extrapolated to 0 kHz (or direct current). The second characteristic resistance value, R∞, is the resistance value obtained when frequency is extrapolated to ∞kHz. These two characteristic resistance values are used in the Cole model with Hanai mixing to determine an individual's total body water (TBW) and subsequently fat free mass (FFM) and fat mass (FM). Total body water is composed of extracellular water (ECW) and intracellular water (ICW). The method for determining ECW utilizes equation (13). In this equation k_{ecf} is an empirical defined constant, the individuals' height and weight are described by Ht and Wt respectively, and R_{ecf} is equivalent to R₀ determined from the reactance versus resistance plot.

ECW=k_{ecf}(Wt^{1/2}Ht²/R_{ecf})^{2/3} (13)

The method for determining ICW utilizes equation (14) and equation (15). In these empirical equations, ECW is determined by equation (13) and r_{IE} is determined using equation (15). In equation (15), r_{LH} is the ration of R_{ecf} to R_{icf}, which are the estimated resistances of ECW and ICW respectively. The resistance of ECW, R_{ecf}, is described above and the resistance of the intracellular fluid is assumed to be the linear combination of R₀ and R_{∞} and is defined as R_{icf}. The constant, kₚ is empirically determined.

ICW=r_{IE}ECW (14)

(1+r_{IE})^{5/2}=r_{LH[}1+(r_{IE}kₚ)] (15)

Combined, ECW and ICW are an individual's TBW. TBW is converted to FFM using the empirical determined conversion of FFM=TBW/0.73. Fat mass is determined by subtracting FFM from total body mass.

Equation (1) is modified below to include a summation of I(t)-E(t) and U(t) over a statistically significant time period t_{sc}:

$${\sum }_{t = {t}_{0}}^{{t}_{sc}} ( I ( t ) - E ( t ) ) = {\sum }_{t = {t}_{0}}^{{t}_{sc}} U ( t ) \qquad ( 16 )$$

The time component t_{sc} can be described as follows: 1) the time an individual starts monitoring caloric intake I(t), caloric expenditure E(t) and stored caloric value U(t) is described by t₀, and 2) the time required to observe a statistical change in body composition during a monitoring or calibration cycle is t_{sc}. This timescale is typically on the order of several days, but can be within a period of hours or weeks, if desired. For example, t₀=1 day on the first day that an individual begins monitoring changes in body composition. If 5 days are required to observe a statistical change in body composition, then t_{sc}=5 days. When changes in body composition are monitored beyond this 5 day period a new t₀ will be defined. In this example, the new t₀=6 days and the new t_{sc}=10 days (assuming the same time required to determine a statistical change in body composition).

According to equation (16), the difference between caloric intake I(t) and caloric expenditure E(t) over a statistically significant period is equal to a change in the stored caloric value U(t) for that period. The left side of equation (16) is termed “net caloric value” herein, and its component variables are discussed in Part I above. The right side of equation (16) relates to a body composition change. Where the stored caloric value U(t) is positive, an increase in body composition is expected. Where the stored caloric value U(t) is negative, a decrease in body composition is expected.

As discussed in Part I above, body composition includes both fat mass FM and fat free mass FFM. The relationship between the caloric value U(t) and fat mass FM and fat free mass FFM is set forth in equation (17) below:

$${ρ}_{F M} \cdot \frac{d}{d t} F M ( t ) + {ρ}_{FFM} \cdot \frac{d}{d t} FFM ( t ) = α ( {x}_{1} , {x}_{2} , … {x}_{n} , {t}_{sc} ) \cdot {\sum }_{t = {t}_{0}}^{{t}_{sc}} U ( t ) \qquad ( 17 )$$

From equation (17) above, the change in fat mass FM and fat free mass FFM is related to the stored caloric value U(t) modified by a personal correlation factor α. That is, not all of the stored caloric value U(t) will be converted to a change in body composition. Instead, a percentage of the stored caloric value U(t) is converted to a change in body composition, with that percentage being represented by the personal correlation factor α. absorbed by the body, converted to glucose and other energy sources, and eventually stored to, or drawn from, fat mass FM and fat free mass FFM.

Referring again to equation (17), the change in body composition is converted to an equivalent energy value by multiplying fat mass FM and fat free mass FFM by the respective energy densities p (kcal/g). On the right side of equation (17), the personal correlation factor α is a dimensionless coefficient that is personal to the individual, and is itself a function of a number of variables, represented by x₁, x₂, . . . xₙ, and t_{sc}. Examples of the independent variables for a personal correlation factor α include any of the following: i) age, ii) gender, iii) genetics, iv) insulin sensitivity, v) weight and vi) activity level.

To determine an individual's specific personal correlation factor α, the independent variables, x can be fixed at scalar values for a specific period of time and t_{sc}, is set to the time required to observe a change in FM and FFM. Some of the independent variables, x₁, x₂, . . . , xₙ, may be reset when a dramatic change occurs individual's life. Other independent variables may fixed indefinitely. For example, the scalar value associated with activity level can be reset if a person started to exercise more during the monitoring cycle, whereas the scalar values associated with genetics, age, and race can be fixed indefinitely. Taking these factors into consideration, a specific personal correlation factor α(t_{sc}) is found by rearranging equation (17), resulting in equation (18) below:

$$\frac{( {ρ}_{F M} \cdot \frac{d}{d t} F M ( t ) + {ρ}_{FFM} \cdot \frac{d}{d t} FFM ( t ) )}{{\sum }_{t = {t}_{0}}^{{t}_{sc}} U ( t )} = α ( {t}_{sc} ) \qquad ( 18 )$$

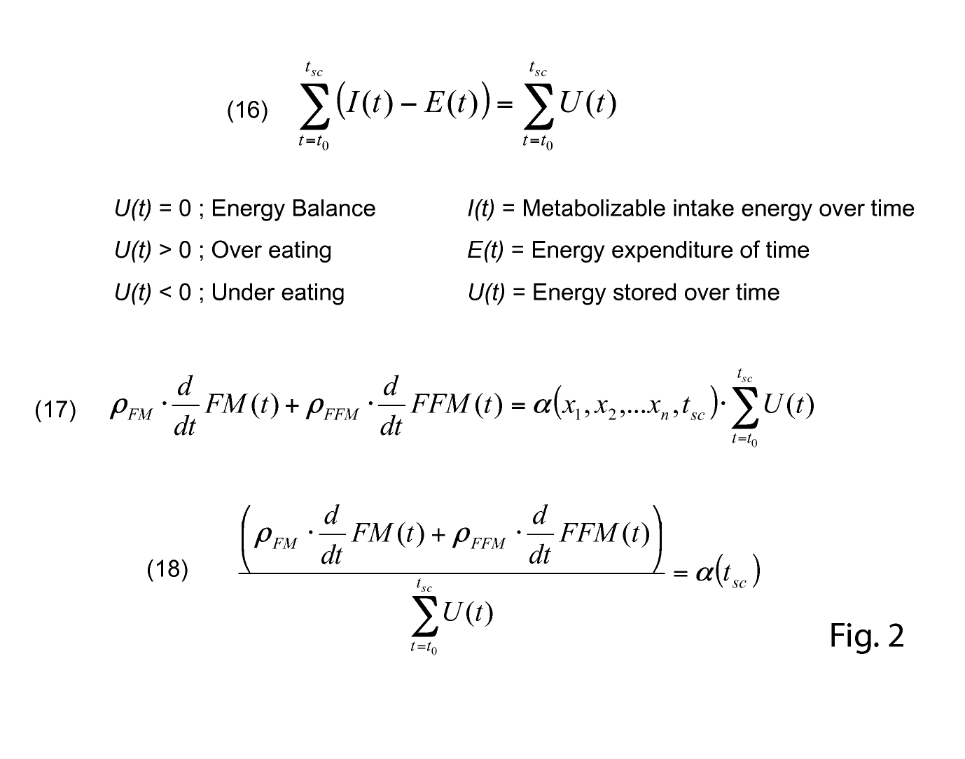

Each of equations (16), (17) and (18) are additionally depicted in FIG. 2. In the ideal case for U(t)<0, every calorie restricted would be removed from either FM or FFM and α(t_{sc})=1. However, this relationship is not predicted to be one-to-one and it is believed that α(t_{sc})>1 in the calorie restricted state.

### II. Determining a Personal Correlation Factor

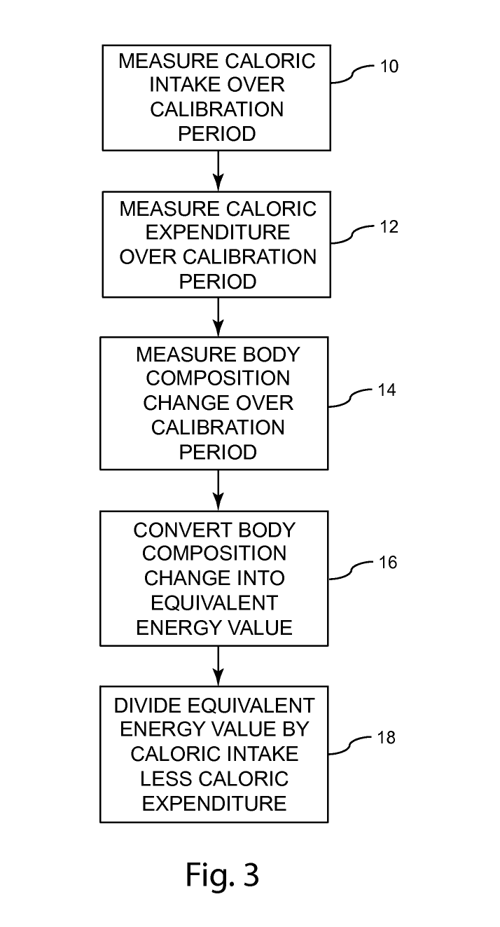

Referring now to the flow chart of FIG. 3, one method for determining a personal correlation factor generally includes measuring a caloric intake over a calibration period at step 10, measuring a caloric expenditure over the calibration period at step 12, measuring a body composition change over the calibration period at step 14, converting the body composition change into an equivalent energy value at step 16, and dividing the equivalent energy value by the caloric intake less the caloric expenditure at step 18.

As the term is used herein, measuring can include any direct or indirect determination or observation of a value, whether the value is estimated, approximated or actual. For example, measuring a caloric intake can include manually tracking a caloric intake over a predefined period of time, and subsequently summing the caloric intake. Also by example, measuring a caloric intake can include providing a meal plan having a plurality of pre-planned meals defining a known number of calories, and quantifying the caloric intake based on the number of meals consumed. More specifically, measuring a caloric intake I(t) at step 10 can be performed in a number of ways. Examples include i) having the individual enter meals into a computer or a device, ii) taking photos of the individual's meals and having a software engine determine or approximate caloric content, iii) scanning a barcode or NFC tag associated with a meal, iv) providing the individual with a pre-package meal plan having a known caloric content and v) combinations of the above. Still other ways for measuring caloric intake I(t) may be used as desired.

Again as noted above, the step of measuring caloric expenditure and body composition can include any direct or indirect determination or observation of an estimated, approximated or actual value. For example, measuring a caloric expenditure E(t) at step 12 can be performed in a number of ways. Examples include i) wearing a device including a three-axis accelerometer to track NEAT AIE, ii) wearing a temperature sensor to track TEF, and iii) taking periodic VO₂/CO₂ measurements to measure BMR. More invasive methods for determining caloric expenditure E(t) include nitrogen balance methods and heavy water techniques. Still other ways for measuring caloric expenditure E(t) may be used as desired. Measuring a change in body composition at step 14 can also be performed in a number of ways. Examples include i) bio-impedance spectroscopy, ii) a mobile scale that can provide weight information and/or bio-impedance measurements and iii) underwater weighing and water displacement tests. Still other ways for measuring a change in body composition may be used as desired.

Once the caloric intake I(t), caloric expenditure E(t) and change in body composition are measured in steps 10 through 14, the individual's actual or approximated personal correlation factor α(t_{sc}) can be determined by computer operation using equation (18) above. In particular, the computer operation can include converting the body composition change into an equivalent energy value at step 16, and dividing this value by the caloric intake I(t) less the caloric expenditure E(t) at step 18. The resulting quotient provides the individual's actual or approximated personal correlation factor α(t_{sc}), which can be used for a number of purposes as set forth more fully in Part III below, including to determine the individual's caloric intake.

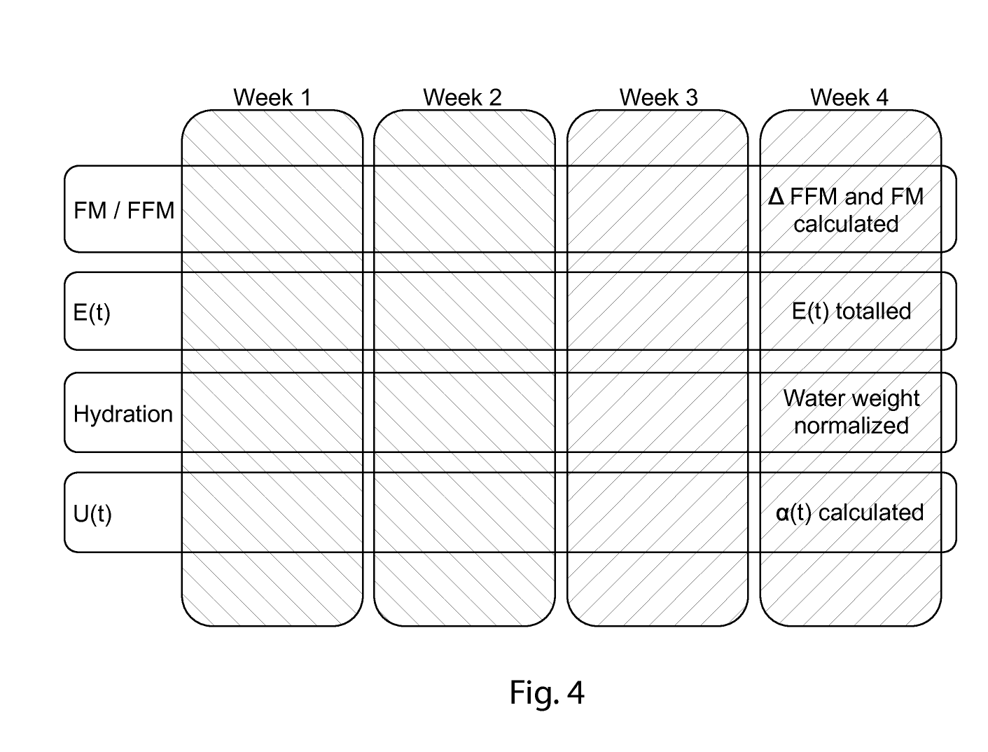

Referring now to FIG. 4, a four-week calibration log for determining an actual or approximated personal correlation factor α(t_{sc}) is illustrated. The calibration log includes weekly entries for fat mass FM and fat free mass FFM, caloric expenditure E(t), hydration level, and stored energy value U(t). During the first two week period, the individual is provided pre-planned meals to provide a known caloric intake I(t). Baseline values are thereby developed for each row in FIG. 4. During the second two week period, the individual is provided with pre-planned meals having a different caloric intake I(t). For example, the caloric intake I(t) can include a 20% reduction in calories. At the conclusion of this second two week period, the change in fat mass FM and fat free mass FFM is determined by subtracting the week-four body composition from the week-two body composition. The change in hydration is also optionally performed to provide a more accurate fat free mass FFM measurement, e.g., to ensure the fat free mass FFM measurement does not include excess fluids. The individual's actual or approximated personal correlation factor α(t_{sc}) can thereafter be determined using a computer operation implementing equation (18) above. In particular, the computer operation can include converting the change in fat mass FM and fat free mass FFM into an equivalent energy value at step 16 of FIG. 3, and dividing this value by the caloric intake I(t) less the caloric expenditure E(t) for weeks three and four at step 18 of FIG. 3.

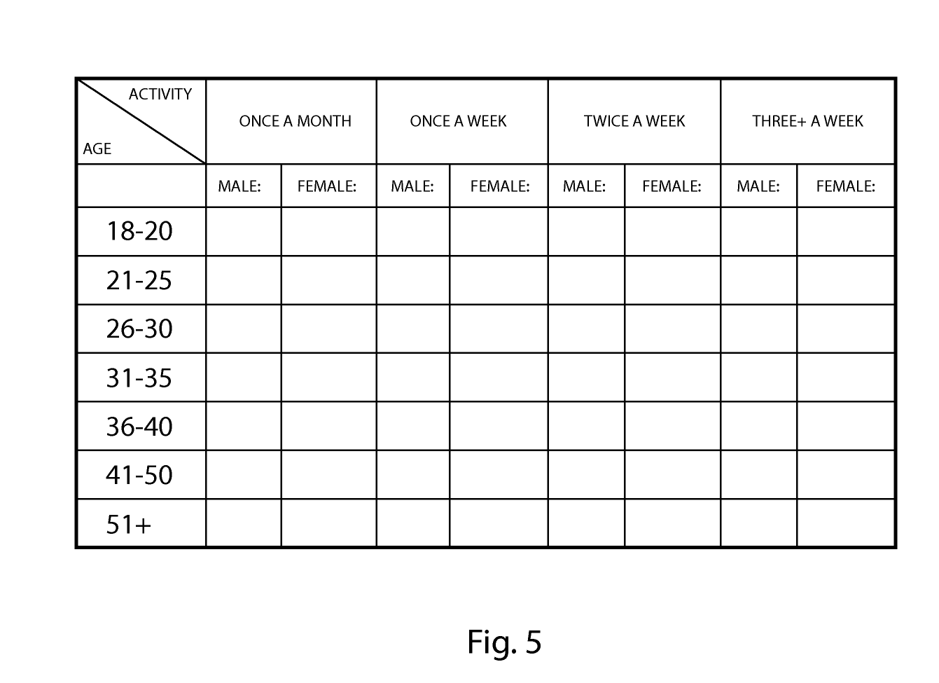

Another method for determining a personal correlation factor α(t_{sc}) includes the collection of clinical data relating to the effects of the independent variables factor α(t_{sc}) for an individual or group of individuals sharing the same physiological or behavioral patterns or characteristics. According to this method for determining a personal correlation factor α(t_{sc}), an individual can input characteristics into a processing engine, including for example a smartphone, a tablet computer, a laptop computer, or other computing device. The processing engine can then determine an actual or approximated personal correlation factor α(t_{sc}) using a lookup table stored to computer readable memory. For example, FIG. 5 illustrates an exemplary look-up table for personal correlation values by age, gender, and workout frequency. Other personal or physiological data can also be used as desired, including for example dietary habits, genetic predispositions, and/or other personal or physiological data. Further by example, the processing engine can determine an actual or approximated personal correlation factor α(t_{sc}) by performing an operation in accordance with a formula for α(t_{sc}).

To reiterate, the present invention provides systems and methods for determining an actual or approximated personal correlation factor α(t_{sc}). One such method includes determining a body composition change over a calibration period, converting the body composition change to an equivalent energy value, and dividing the equivalent energy value by a net caloric value for the same calibration period, wherein the net caloric value includes the individual's caloric expenditure less the individual's caloric intake. Another such method includes aggregating physiological data pertaining to the individual, and determining an actual or approximated personal factor with reference to a lookup table and/or a numerical computer operation.

Because the personal correlation factor α(t_{sc}) can be a function of a number of independent variables, the personal correlation factor α(t_{sc}) can periodically be ‘recalibrated,’ for example as the individual experiences significant changes in health, weight, age, stress level, diet, sleep patterns and other conditions. Further by example, the personal correlation factor α(t_{sc}) can be recalibrated at regular intervals in a weight loss or weight management program, or upon reaching certain weight loss milestones. Further by example, the personal correlation factor α(t_{sc}) can be recalibrated on a monthly basis, a semi-annual basis, or an annual basis as part of regular progress checks in a weight loss or weight management program. Still other recalibration intervals can be used as desired.

### III. Determining a Caloric Intake

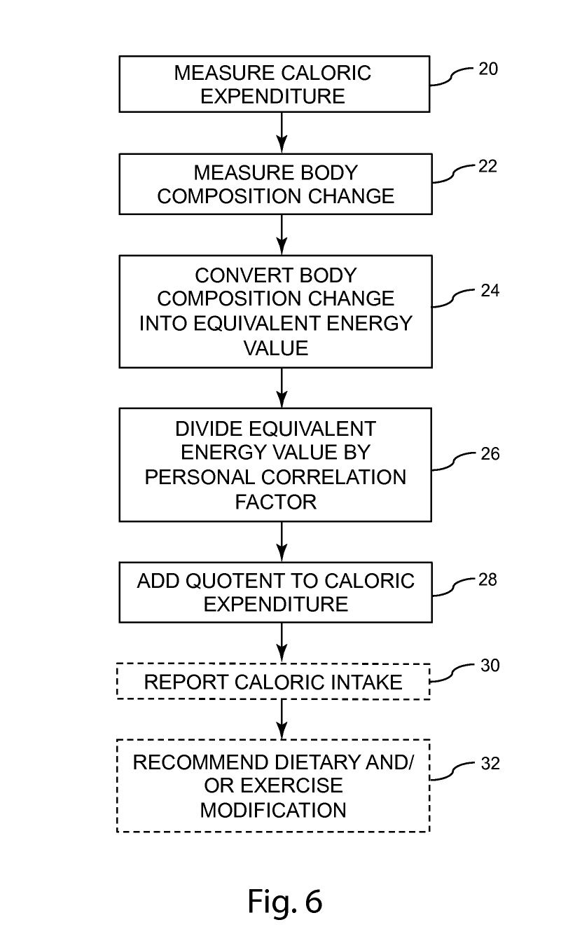

Once the individual's actual or approximated personal correlation factor α(t_{sc}) is determined, the personal correlation factor α(t_{sc}) can be used to indirectly measure an individual's actual or approximated caloric intake I(t). Referring now to the flow chart of FIG. 6, one method for determining a caloric intake I(t) generally includes measuring a caloric expenditure E(t) at step 20, measuring a body composition change at step 22, converting the body composition change into an equivalent energy value at step 24, dividing the equivalent energy value by the individual's actual or approximated personal correlation factor α(t_{sc}) at step 26, and adding to this quotient the individual's caloric expenditure E(t) at step 28, wherein at least steps 24, 26 and 28 are performed using a processor. The method can additionally include reporting the caloric intake at step 30, optionally with reference to a target value. Still further optionally, the method can include recommending a dietary modification and/or recommending an exercise regimen at step 32 and in response to the determined caloric intake.

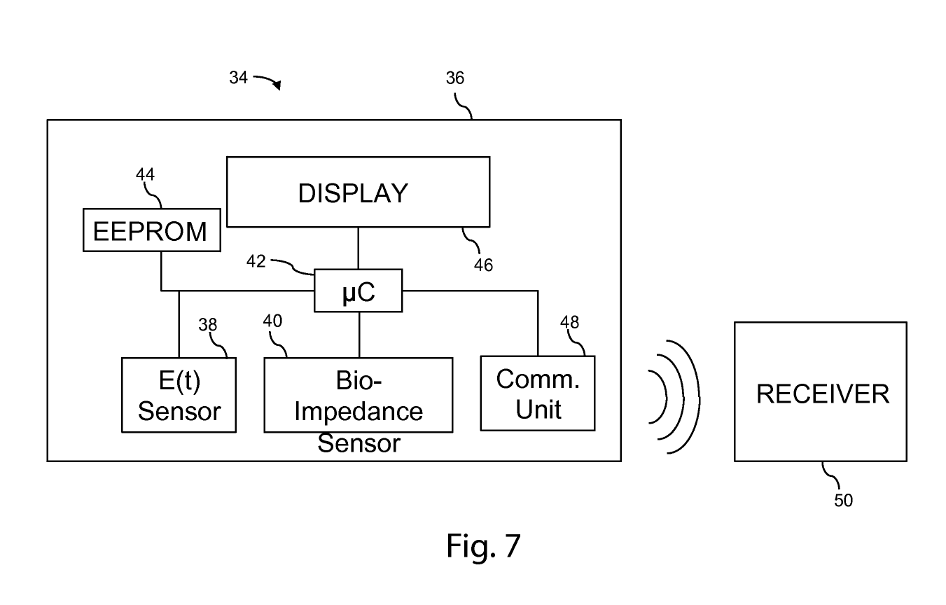

More specifically, measuring a caloric expenditure E(t) at step 20 and measuring a body composition change at step 22 can be performed using a portable device. Referring now to FIG. 7, an exemplary portable device 34 is schematically shown, the portable device 34 including a housing 36, a first sensor 38 for determining a caloric expenditure, a second sensor 40 for determining a change in body composition, a processor 42 electrically coupled to the output of the first and second sensors 36, 38, a memory 44, and a display 46 for presenting the caloric intake and other data to an individual. The processor can additionally include one or more communication units 48 for transmitting information to a central hub or receiver station 50, the information including the individual's caloric expenditure, the individual's change in body composition, or other physiological or personal data. More specifically, the device housing 36 may be in the form of a wearable item, such as a wristband, bracelet, anklet or other similar item. As another example, the housing may be in a form suitable for carrying or clipping to a user's clothing. In any event, it may be desirable to provide a housing that is water-resistant or waterproof

In the present embodiment, the first sensor 38 includes a three-axis accelerometer adapted to detect an input relating to the three-dimensional motion of the host individual. In other embodiments, the first sensor 38 can alternatively include other motion or orientation sensors to determine an actual or approximated energy expenditure E(t). The second sensor 40 includes bio-impedance circuitry adapted to detect an input relating to the fat mass FM and fat free mass FFM of the host individual. The bio-impedance circuitry can include an interior sensor configured to engage the user's skin beneath the device and an exposed sensor that can be placed in contact with the user's skin at a location remote from the interior sensor. For example, if the personal device is a wristband, one sensor may be located on the inside of the wristband to engage the user's wrist on one arm and the other sensor may be exposed on the outside of the wristband so that it can be placed in contact with the skin on the user's other wrist to provide an arm-to-arm bio-impedance measurement. Other body composition measurement sensors can be used in other embodiments as desired. Additional sensors can also be utilized, including for example a temperature sensor or a moisture sensor.

As noted above, the first and second sensors 38, 40 are electrically coupled to the processor 42. The processor can be any processor adapted to perform a program set, including an integrated circuit, a microcontroller, or a field-programmable gate array. For example, the processor 42 can be configured to determine a prior caloric intake based on at least one sensor input and a personal correlation factor. Further by example, the processor 42 can be configured to determine a prior caloric intake based on the first and second sensor inputs and based on a personal correlation factor by implementing method steps 24, 26 and 28 noted above in connection with FIG. 6. This determined caloric intake can be displayed on the display 46, optionally an AMOLED display, an LCD display, an e-ink display, or other display whether now known or hereinafter developed. Alternatively, the display 46 can present the host individual's progress in accordance with a predetermined diet, optionally as part of a larger weight management or fitness regimen.

As noted above, the portable device 34 includes an on-board memory 44 electrically coupled to the processor 42. The onboard memory 44 can be utilized to store one or more values, including for example the values used in the performance of method steps 24, 26 and 28. These values can include, but are not limited to, energy expenditure, body composition, personal correlation factor, and caloric input. The memory includes non-volatile memory in the present embodiment, including for example flash memory or EEPROM, but can include volatile or other categories of memory in other embodiments.

The portable device 34 further optionally includes a communications unit 48 electrically coupled to the processor 42. The communications unit 48 can be any unit adapted to transmit and/or receive wireless communications to or from a receive station 50 over a communications network. Exemplary networks include a Bluetooth network, a WiFi network, and a ZigBee network. Still other networks may be used in other embodiments as desired.

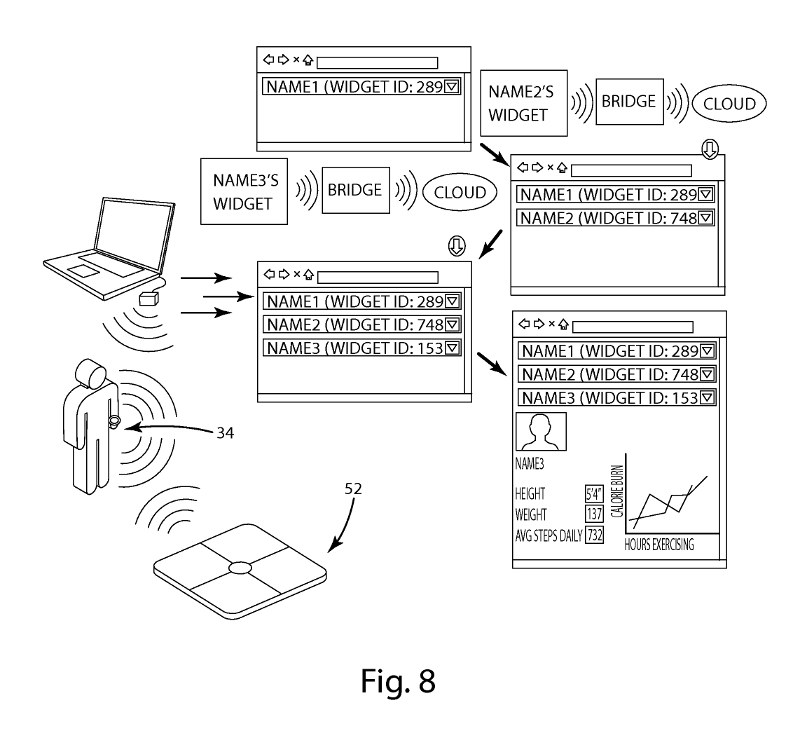

As further optionally shown in FIG. 8, caloric expenditure and/or body composition can be measured using similar worn or carried sensors which push their collected data to a remote processor. The remote processor can process the data remotely to determine the caloric intake, with the results being transmitted back to the user through the same device 34 or through an alternative device, including for example a smartphone, a tablet computer, or a laptop computer. Additionally, information from other remote sensors 52 may also be gathered, such as a scale that measures weight, an energy expenditure device such as a pedometer, a sensor applied on or under the skin, and other sensor types. By calculating the caloric intake, the remote processor can recommend diet changes, exercise programs, nutrition supplements, or other lifestyle changes to encourage positive changes in body composition. For example, the system of FIG. 8 can report the caloric intake with reference to a target value. Still further by example, the system can recommend a dietary modification and/or recommending an exercise regimen in response to the determined caloric intake. Still other information can be communicated to the user using the system of FIG. 8 as generally set forth in International Patent Application PCT/US12/68503 filed Dec. 7, 2012, and entitled Behavior Tracking and Modification System, the disclosure of which is hereby incorporated by reference in its entirety.

Directional terms, such as “vertical,” “horizontal,” “top,” “bottom,” “upper,” “lower,” “inner,” “inwardly,” “outer” and “outwardly,” are used to assist in describing the invention based on the orientation of the embodiments shown in the illustrations. The use of directional terms should not be interpreted to limit the invention to any specific orientation(s).

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits.

The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

1. A method comprising:
measuring a caloric intake;
measuring a caloric expenditure;
measuring a change in body composition;
performing an operation, using a computer, to determine a personal correlation factor based on the caloric intake, the caloric expenditure, and the change in body composition; and
reporting the personal correlation factor as an output.

2. The method according to claim 1 wherein the operation includes:
converting the change in body composition into an equivalent energy value, and
dividing the equivalent energy value by the measured caloric intake less the measured caloric expenditure.

3. The method according to claim 1 further including utilizing the personal correlation factor to determine a subsequent caloric intake.

4. The method according to claim 3 wherein the caloric intake includes a macronutrient intake.

5. The method according to claim 4 further including determining, using the computer, the macronutrient contribution to the individual's change in body composition.

6. The method according to claim 1 further including providing a first meal plan having a caloric content, wherein the measured caloric intake includes the caloric content of the first meal plan.

7. The method according to claim 6 further including providing a second meal plan having a caloric content less than the first meal plan caloric content to generate a measurable change in body composition.

8. A method comprising:
providing a computer-based input device;
accepting an individual's biometric data to the computer-based input device; and
deriving, using the computer-based input device, a personal correlation factor based on the biometric data and a look-up table stored in computer readable memory.

9. The method according to claim 8 wherein the individual's biometric data is selected from the group consisting of the individual's age, gender, workout regimen, blood-sugar level, diet, height, genetic markers, weight, smoking habits, alcohol consumption, prescription drug usage and combinations thereof.

10. The method according to claim 8 further including:
measuring the individual's caloric expenditure;
measuring the individual's change in body composition; and
determining a caloric intake based on the measured caloric expenditure, the measured change in body composition, and the personal correlation factor.

11. The method according to claim 10 wherein measuring a caloric expenditure is performed using an accelerometer.

12. The method according to claim 10 wherein measuring a change in body composition includes measuring a bio-impedance.

13. The method according to claim 10 wherein the change in body composition includes a reduction in fat mass or fat free mass.

14. The method according to claim 10 further including recommending an activity or an exercise in response to the caloric intake determination.

15. The method according to claim 10 further including recommending a dietary modification in response to the caloric intake determination.

16. A method for determining a caloric intake comprising:
measuring a caloric expenditure;
measuring a change in body composition; and
determining a caloric intake based on the measured caloric expenditure, the measured change in body composition, and a personal correlation factor.

17. The method according to claim 16 wherein measuring the caloric expenditure includes measuring ambulatory motion.

18. The method according to claim 16 wherein measuring the caloric expenditure includes measuring arm motion.

19. The method according to claim 16 wherein measuring a caloric expenditure is performed using an accelerometer.

20. The method according to claim 16 wherein measuring a change in body composition includes measuring a bio-impedance.

21. The method according to claim 16 wherein the change in body composition includes a reduction in fat mass or fat free mass.

22. The method according to claim 16 wherein the personal correlation factor is based on a ratio of a prior caloric deficiency and a prior change in body composition.

23. The method according to claim 22 wherein the prior caloric deficiency includes a prior caloric expenditure less a prior caloric intake for a given period of time.

24. The method according to claim 14 further including recommending an activity or an exercise in response to the caloric intake determination.

25. The method according to claim 14 further including recommending a dietary modification in response to the caloric intake determination.

26. A wearable device comprising:
a sensor configured to detect an input; and
a processor electrically coupled to the sensor and adapted to determine a prior caloric intake, wherein the prior caloric intake determination is based on the sensor input and a personal correlation factor.

27. The device according to claim 26 wherein the sensor is adapted to measure energy expenditure.

28. The device according to claim 27 wherein the sensor includes at least one of a pedometer and an accelerometer.

29. The device according to claim 26 wherein the sensor is adapted to measure bio-impedance.

30. The device according to claim 29 wherein the sensor is adapted to determine body composition based on the measured bio-impedance.

31. The device according to claim 26 wherein the personal correlation factor is based on a ratio of a prior caloric deficiency and a prior change in body composition.

32. The device according to claim 31 wherein the prior caloric deficiency includes a caloric expenditure less a caloric intake for a given period of time.

33. The device according to claim 31 wherein the prior change in body composition includes a reduction in fat mass and fat free mass.

34. The device according to claim 26 wherein the processor is adapted to select a recommended activity or exercise in response to the caloric intake determination.

35. The device according to claim 26 wherein the processor is adapted to select a recommended dietary modification in response to the caloric intake determination.

36. A system comprising:
a plurality of sensors configured to detect a first input relating to an individual's caloric expenditure, a second input relating to the individual's change in body composition, and a third input relating to the individual's hydration level; and
a processor electrically coupled to the plurality of sensors and adapted to determine physiological information pertaining to the individual based on the input for each of the plurality of sensors and a personal correlation factor.

37. The system of claim 36 wherein the physiological information includes at least one of caloric intake, caloric expenditure and body composition.

38. The system of claim 36 wherein the processor is coupled to the plurality of sensors over a wireless network.

39. The system of claim 36 wherein the processor is separate from the plurality of sensors.

40. The system of claim 36 wherein the first plurality of sensors include at least one of a pedometer, an accelerometer, and a bio-impedance sensor.

41. A system comprising:
a first plurality of sensors configured to detect a first plurality of inputs relating to a first individual's physiology;
a second plurality of sensors configured to detect a second plurality of inputs relating to a second individual's physiology; and
a processor electrically coupled to the first and second plurality of sensors and adapted to determine a personal correlation factor for subsequent use by the first and second individuals to determine a caloric intake for the first and second individuals.

42. The system of claim 41 wherein the processor is coupled to the first and second plurality of sensors over a wireless network.

43. The system of claim 41 wherein each of the first and second plurality of sensors include a pedometer, an accelerometer, or a bio-impedance sensor.

44. The system of claim 41 wherein the first individual's physiology includes the first individual's caloric intake, caloric expenditure, and body composition.

45. The system of claim 41 wherein the second individual's physiology includes the second individual's caloric intake, caloric expenditure, and body composition.

46. The system of claim 41 further including a computer readable memory including a look-up table, the look-up table including the personal correlation factor.

47. The system of claim 46 wherein the processor is adapted to reference the personal correlation factor stored in the computer readable memory to determine a caloric intake for a third individual.
